(19) United States
(12) Patent Application Publication
(10) Pub. No.: US 2020/0077969 A1
(43) Pub. Date: Mar. 12, 2020

(54) MEDICAL IMAGING DEVICE AND MEDICAL IMAGE PROCESSING METHOD

(71) Applicant: Samsung Electronics Co., Ltd., Suwon-si, Gyeonggi-do (KR)

(72) Inventors: Kyoung-yong LEE, Hwaseong-si (KR); Dong-gue LEE, Seongnam-si (KR)

(73) Assignee: Samsung Electronics Co., Ltd., Suwon-si, Gyeonggi-do (KR)

(21) Appl. No.: 16/465,905

(22) PCT Filed: Jul. 13, 2017
(86) PCT No.: PCT/KR2017/007491

§ 371 Date: May 31, 2019

(30) Foreign Application Priority Data
Dec. 23, 2016 (KR) 10-2016-0177942

(51) Int. Cl. A61B 6/00; A61B 6/03; G06T 11/00

(52) U.S. Cl. CPC A61B 6/5205; A61B 6/5264; G06T 2210/41; A61B 6/465; G06T 11/008; A61B 6/032

(57) ABSTRACT

Provided are a medical imaging device and a medical image processing method. A medical imaging device includes a data obtainer configured to obtain raw data by performing computed tomography (CT) imaging on an object; a processor configured to set a plurality of regions based on the raw data or an image generated from the raw data, determine at least one reconstruction processing method for each of the plurality of regions, and reconstruct a CT image by applying the determined at least one reconstruction processing method to each of the plurality of regions; and a display displaying the reconstructed CT image.

### TECHNICAL FIELD

The present disclosure relates to a device and a method for processing a medical image, and a computer-readable recording medium having recorded thereon a program code for executing the method.

### BACKGROUND ART

A medical imaging device is a device for displaying an internal structure of an object as an image. The medical imaging device is a non-invasive test device which captures and processes images of structural details, internal tissues, and fluid flow in an object and displays the same to a user. The user, such as a doctor, may diagnose a health condition and disease of a patient by using a medical image output from the medical imaging device. The characteristics of the medical image may vary depending on a region of a captured object, and accordingly, a required image processing method may vary for each region of the object. Accordingly, in order to obtain a medical image of image quality desired by the user more quickly and effectively, a method of applying different image processing methods to each region of the object is needed.

### DESCRIPTION OF EMBODIMENTS

### Technical Problem

Provided is reconstruction of a computed tomography (CT) image of image quality desired by a user more effectively by applying different reconstruction processing methods according to a region of an object.

Provided is reconstruction of a CT image of image quality desired by a user more quickly by applying different reconstruction processing methods in parallel according to a region of an object.

### Solution to Problem

A medical imaging device may include a data obtainer configured to obtain raw data by performing computed tomography (CT) imaging on an object; a processor configured to set a plurality of regions based on the raw data or an image generated from the raw data, determine at least one reconstruction processing method for each of the plurality of regions, and reconstruct a CT image by applying the determined at least one reconstruction processing method to each of the plurality of regions; and a display displaying the reconstructed CT image.

### BRIEF DESCRIPTION OF DRAWINGS

FIG. 1 illustrates a structure of a computed tomography (CT) system according to an embodiment.

FIG. 2 is a block diagram illustrating a configuration of a medical imaging device according to an embodiment.

FIGS. 3A and 3B are diagrams for explaining a process of setting a plurality of regions, according to an embodiment.

FIGS. 4A to 4C are diagrams for explaining a process of setting a plurality of regions performed by a medical imaging device, according to an embodiment.

FIG. 5 is a diagram for explaining a process of automatically setting a plurality of regions performed by a medical imaging device, according to an embodiment.

FIGS. 6A to 6D are diagrams for explaining a process of setting a parameter of a reconstruction processing method differently according to an embodiment.

FIG. 7 is a diagram for explaining a process of manually selecting a reconstruction processing algorithm applied to each of a plurality of regions, according to an embodiment.

FIG. 8 is a diagram for explaining a process of applying at least one reconstruction processing method to each of a plurality of regions, according to an embodiment.

FIG. 9 is a flowchart illustrating a medical image processing method according to an embodiment.

### BEST MODE

A medical imaging device may include a data obtainer configured to obtain raw data by performing computed tomography (CT) imaging on an object; a processor configured to set a plurality of regions based on the raw data or an image generated from the raw data, determine at least one reconstruction processing method for each of the plurality of regions, and reconstruct a CT image by applying the determined at least one reconstruction processing method to each of the plurality of regions; and a display displaying the reconstructed CT image.

The processor may be further configured to determine the at least one reconstruction processing method differently for each of the plurality of regions.

The display may further display a user interface representing at least one of a type or a parameter of a reconstruction processing algorithm provided by the medical imaging device, and the processor may be further configured to receive, through the user interface, an input for selecting the at least one reconstruction processing algorithm for each of the plurality of regions.

The at least one reconstruction processing method may include at least one of a streak artifact reduction method, a motion artifact reduction method, a metal artifact reduction method, a noise reduction method, and a resolution improvement method, or a combination thereof.

The processor may be further configured to automatically set the plurality of regions based on anatomical characteristics of the object.

The processor may be further configured to determine, for each of the plurality of regions, at least one reconstruction processing algorithm corresponding to the at least one reconstruction processing method, and, for each of the plurality of regions, apply the determined at least one reconstruction processing algorithm to reconstruct the CT image from the raw data.

The processor may be further configured to automatically determine the at least one reconstruction processing method according to a predetermined criterion.

The processor may be further configured to change the at least one automatically determined reconstruction processing method in response to an external input.

The processor may be further configured to receive an input for selecting at least one reconstruction processing algorithm for each of the plurality of regions, and, in response to the received input, reconstruct the CT image using the selected at least one reconstruction processing algorithm.

The processor may be further configured to perform reconstruction processing on each of the plurality of regions in parallel to reconstruct the CT image.

A medical image processing method may include obtaining raw data by performing computed tomography (CT) imaging on an object; setting a plurality of regions based on the raw data or an image generated from the raw data; determining at least one reconstruction processing method for each of the plurality of regions; reconstructing a CT image by applying the determined at least one reconstruction processing method to each of the plurality of regions; and displaying the reconstructed CT image.

### MODE OF DISCLOSURE

The principle of the present invention is explained and embodiments are disclosed so that the scope of the present invention is clarified and one of ordinary skill in the art to which the present invention pertains implements the present invention. The disclosed embodiments may have various forms.

Throughout the specification, like reference numerals or characters refer to like elements. In the present specification, all elements of embodiments are not explained, but general matters in the technical field of the present invention or redundant matters between embodiments will not be described. Terms ‘module’ or ‘unit’ used herein may be implemented using at least one or a combination from among software, hardware, or firmware, and, according to embodiments, a plurality of ‘module’ or ‘unit’ may be implemented using a single element, or a single ‘module’ or ‘unit’ may be implemented using a plurality of units or elements. The operational principle of the present invention and embodiments thereof will now be described more fully with reference to the accompanying drawings.

In the present specification, an image may include a medical image obtained by a medical imaging device, such as a computed tomography (CT) device, a magnetic resonance imaging (MRI) device, an ultrasound imaging device, or an X-ray device.

Throughout the specification, the term ‘object’ is a thing to be imaged, and may include a human, an animal, or a part of a human or animal. For example, the object may include a part of a body (i.e., an organ), a phantom, or the like.

In the present specification, a ‘CT system’ or ‘CT device’ refers to a system or device configured to emit X-rays while rotating around at least one axis relative to an object and photograph the object by detecting the X-rays.

In the specification, a ‘CT image’ refers to an image constructed from raw data obtained by imaging an object by detecting X-rays that are emitted as the CT system or device rotates about at least one axis with respect to the object.

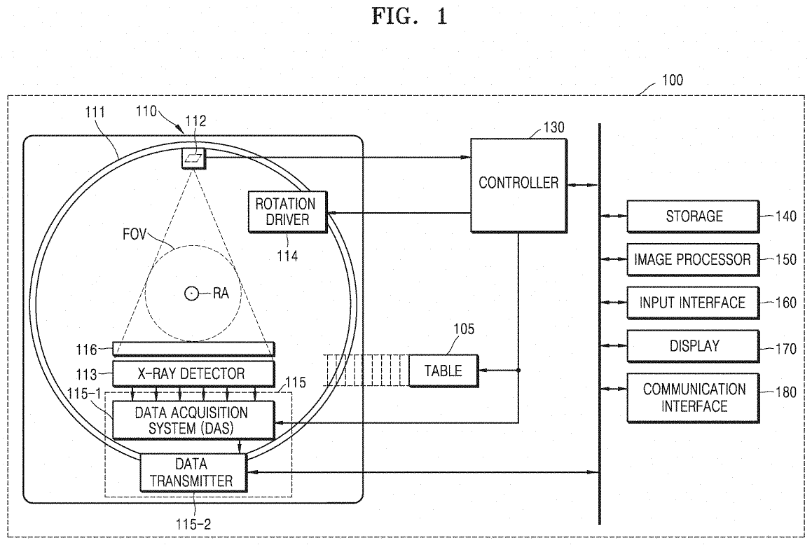

FIG. 1 illustrates a structure of a CT system 100 according to an embodiment.

The CT system 100 may include a gantry 110, a table 105, a controller 130, a storage 140, an image processor 150, an input interface 160, a display 170, and a communication interface 180.

The gantry 110 may include a rotating frame 111, an X-ray generator 112, an X-ray detector 113, a rotation driver 114, and a readout device 115.

The rotating frame 111 may receive a driving signal from the rotation driver 114 and rotate around a rotation axis (RA).

An anti-scatter grid 116 may be disposed between an object and the X-ray detector 113 and may transmit most of primary radiation and attenuate scattered radiation. The object may be positioned on the table 105 which may move, tilt, or rotate during a CT scan.

The X-ray generator 112 receives a voltage and a current from a high voltage generator (HVG) to generate and emit X-rays.

The CT system 100 may be implemented as a single-source CT system including one X-ray generator 112 and one X-ray detector 113, or as a dual-source CT system including two X-ray generators 112 and two X-ray detectors 113.

The X-ray detector 113 detects radiation that has passed through the object. For example, the X-ray detector 113 may detect radiation by using a scintillator, a photon counting detector, etc.

Methods of driving the X-ray generator 112 and the X-ray detector 113 may vary depending on scan modes used for scanning of the object. The scan modes are classified into an axial scan mode and a helical scan mode, according to a path along which the X-ray detector 113 moves. Furthermore, the scan modes are classified into a prospective mode and a retrospective mode, according to a time interval during which X-rays are emitted.

The controller 130 may control an operation of each of the components of the CT system 100. The controller 130 may include a memory configured to store program for performing a function or data and a processor configured to process the program codes or the data. The controller 130 may be implemented in various combinations of at least one memory and at least one processor. The processor may generate or delete a program module according to an operating status of the CT system 100 and process operations of the program module.

The readout device 115 receives a detection signal generated by the X-ray detector 113 and outputs the detection signal to the image processor 150. The readout device 115 may include a data acquisition system (DAS) 115-1 and a data transmitter 115-2. The DAS 115-1 uses at least one amplifying circuit to amplify a signal output from the X-ray detector 113, and outputs the amplified signal. The data transmitter 115-2 uses a circuit such as a multiplexer (MUX) to output the signal amplified in the DAS 115-1 to the image processor 150. According to a slice thickness or a number of slices, only some of a plurality of pieces of data collected by the X-ray detector 113 may be provided to the image processor 150, or the image processor 150 may select only some of the plurality of pieces of data.

The image processor 150 obtains tomography data from a signal obtained by the readout device 115 (e.g., pure data that is data before being processed). The image processor 150 may pre-process the obtained signal, convert the obtained signal into tomography data, and post-process the tomography data, The image processor 150 may perform some or all of the processes described herein, and the type or order of processes performed by the image processor 150 may vary according to embodiments.

The image processor 150 may perform pre-processing, such as a process of correcting sensitivity irregularity between channels, a process of correcting a rapid decrease of signal strength, or a process of correcting signal loss due to an X-ray absorbing material, on the signal obtained by the readout device 115.

According to embodiments, the image processor 150 may perform some or all of the processes for reconstructing a tomography image, to thereby generate the tomography data. According to an embodiment, the tomography data may be in the form of data that has undergone back-projection, or in the form of a tomography image. According to embodiments, additional processing may be performed on the tomography data by an external device such as a server, a medical device, or a portable device.

The CT system 100 performs tomographic imaging on the object to acquire raw data to obtain the tomography image. The CT system 100 generates X-rays, irradiates the X-rays to an object, and uses the X-ray detector 113 to detect X-rays passing through the object. The X-ray detector 113 generates raw data corresponding to the detected X-rays. The raw data may refer to data before being reconstructed as the tomography image by the image processor 150. The raw data is a set of data values corresponding to intensities of X-rays that have passed through the object, and may include projection data or a sinogram. The data that has undergone back-projection is obtained by performing back-projection on the raw data by using information about an angle at which X-rays are emitted. The tomography image is obtained by using image reconstruction techniques including back-projection of the raw data.

The storage 140 is a storage medium for storing control-related data, image data, etc., and may include a volatile or non-volatile storage medium.

The input interface 160 receives control signals, data, etc., from a user. The display 170 may display information indicating an operational status of the CT system 100, medical information, medical image data, etc.

The CT system 100 includes the communication interface 180 and may be connected to external devices, such as a server, a medical device, and a portable device (smartphone, tablet personal computer (PC), wearable device, etc.), via the communication interface 180.

The communication interface 180 may include one or more components that enable communication with an external device. For example, the communication interface 180 may include at least one of a short distance communication module, a wired communication module, and a wireless communication module.

The communication interface 180 may receive control signals and data from an external device and transmit the received control signals to the controller 130 such that the controller 130 controls the CT system 100 according to the received control signals.

Alternatively, the controller 130 may transmit control signals to the external device through the communication interface 180, thereby controlling the external device according to the control signals of the controller 130.

For example, the external device may process data of the external device according to the control signals of the controller 130 received through the communication interface 180.

The external device may be provided with a program capable of controlling the CT system 100. The program may include an instruction to perform a part or all of the operation of the controller 130.

The program may be installed in advance in an external device, or a user of the external device may download and install the program from a server providing an application. The server providing the application may include a recording medium storing the program.

According to embodiments, the CT system 100 may or may not use contrast media during a CT scan, and may be implemented as a device connected to other equipment.

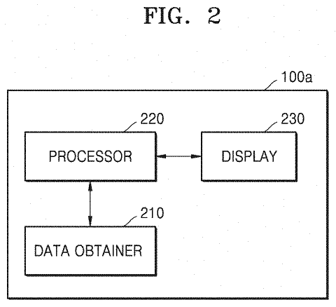

FIG. 2 is a block diagram illustrating a configuration of a medical imaging device according to an embodiment.

The medical imaging device according to an embodiment is an device for processing and displaying medical image data and may be implemented in the form of an electronic device. For example, the medical imaging device 100a may be implemented as various types of devices including a processor and a display, such as a general-purpose computer, a tablet PC, a smart phone, and the like. Also, the medical imaging device according to an embodiment may be implemented as the CT system 100 shown in FIG. 1.

Referring to FIG. 2, the medical imaging device 100a according to an embodiment may include a data obtainer 210, a processor 220, and a display 230. However, the medical imaging device 100a may be implemented by more elements than the illustrated elements and is not limited to the above-described example.

The data obtainer 210 according to an embodiment may obtain raw data generated by performing CT imaging on an object. The raw data may be obtained in various manners, such as obtaining from a scanner of the medical imaging device 100a, receiving from an external device, or the like.

According to an embodiment, the data obtainer 210 may correspond to the scanner of the medical imaging device 100a and may include, for example, the gantry 110 of the CT system 100 shown in FIG. 1. Accordingly, the data obtainer 210 may include a rotating frame 111, an X-ray generator 112, an X-ray detector 113, a rotation driver 114, and the readout device 115 shown in FIG. 1.

According to another embodiment, the data obtainer 210 may be implemented in the form of a communicator communicating with an external device. The data obtainer 210 may receive the raw data obtained by imaging the object from the external device.

The processor 220 performs predetermined processing based on a received user input. The processor 220 may be implemented in various combinations of one or more memories and one or more processors. For example, a memory may generate and delete a program module according to an operation of the processor 220 and the processor 220 may process operations of the program module.

The processor 220 according to an embodiment sets a plurality of regions based on the raw data obtained through the data obtainer 210 or an image generated from the raw data.

The plurality of regions may be regions requiring different reconstruction processing methods. According to an embodiment, the plurality of regions may be regions that are distinguished according to the anatomical characteristics of an object. For example, the processor 220 may segment the organs of the human body to establish the plurality of regions. For example, the processor 220 may set regions representing the shoulders, the heart, and the lungs as different regions. At this time, because components constituting each organ of the human body are different from each other and the characteristics of a region including a metal are different, the characteristics of a CT image corresponding to each of the plurality of regions may be different. Alternatively, when the metal is included in the object, the processor 220 may set a region including the metal as one region.

Also, the processor 220 according to an embodiment may automatically set the plurality of regions based on the anatomical characteristics of the object.

The processor 220 according to an embodiment determines at least one reconstruction processing method for each of the plurality of regions. For example, the reconstruction processing method may include a streak artifact reduction method, a motion artifact reduction method, a metal artifact reduction method, a resolution improvement method, a noise reduction method, etc. and each reconstruction processing method may be implemented by various algorithms. For example, the noise reduction method may include algorithm applied to raw data prior to reconstructing the CT image, algorithm applied in reconstructing the CT image, and algorithm applied to the reconstructed the CT image.

Also, the processor 220 may apply a different reconstruction kernel for each of the plurality of regions. For example, the processor 220 may apply a sharper kernel to a region where the internal structure or boundary of the object should appear more clearly. Unlike this, the processor 220 may apply a smoother kernel to a region that needs to reduce a noise level.

The processor 220 according to an embodiment may determine the application strength of the at least one reconstruction processing method applied to each of the plurality of regions. For example, the processor 220 may determine a parameter of the at least one reconstruction processing method applied to each of the plurality of regions based on at least one of a degree of the noise level, an occurrence degree of a motion artifact, an occurrence degree of a streak artifact, or an occurrence degree of a metal artifact.

The processor 220 according to an embodiment may determine, for each of the plurality of regions, at least one reconstruction processing algorithm for applying the at least one reconstruction processing method. As described above, each reconstruction processing method may be implemented by various algorithms. For example, the processor 220 may provide various algorithms for applying each of the streak artifact reduction method, the metal artifact reduction method, the motion artifact reduction method, the noise reduction method, and the resolution improvement method.

For example, the processor 220 may provide a statistical weighting algorithm that reduces streak artifacts by setting weights differently depending on the statistical characteristics of the raw data, as an algorithm corresponding to the streak artifact reduction method. As another example, the processor 220 may provide, as an algorithm corresponding to the motion artifact reduction method, an algorithm that reduces motion artifacts by measuring motion of the object based on a non-rigid registration method, an algorithm that warps pixels in a backprojection step based on an expected motion of the object, and the like, but is not limited to the example described above.

The processor 220 according to an embodiment may determine one of at least one algorithm included in a specific reconstruction processing method to apply the specific reconstruction processing method. At this time, the processor 220 may automatically determine one of the at least one algorithm according to the initial setting of the medical imaging device 100a. According to an embodiment, the processor 220 may determine one of the at least one algorithm based on the preference of a user.

The processor 220 according to an embodiment may receive, for each of the plurality of regions, an input for selecting at least one reconstruction processing algorithm. For example, the processor 220 may determine at least one reconstruction processing method for each of the plurality of regions, and control the display 230 to display various reconstruction processing algorithm lists corresponding to each of the at least one reconstruction processing method. The processor 220 may receive, for each of the plurality of regions, an input for selecting one of the various reconstruction processing algorithm lists corresponding to each of the at least one reconstruction processing method. Accordingly, the processor 220 may determine the reconstruction processing algorithm applied to each of the plurality of regions in consideration of the preference of the user.

The processor 220 according to an embodiment may change the at least one automatically determined reconstruction processing method in response to an external input. As described above, the processor 220 may automatically determine the at least one reconstruction processing method for each of the plurality of regions. However, even when the reconstruction processing method is automatically determined, the processor 220 may allow the user to change at least one of a type or a parameter of the reconstruction processing method applied to each of the plurality of regions as needed.

The processor 220 according to an embodiment reconstructs a CT image for each of the plurality of regions from the raw data by applying the determined reconstruction processing method. The processor 220 may reconstruct the CT image more quickly by performing reconstruction processing on each of the plurality of regions in parallel and reconstructing the CT image.

The display 230 according to an embodiment displays the CT image reconstructed by the processor 220.

When the display 230 is implemented as a touch screen, the display 230 may be used as an input device in addition to an output device. The display 230 may be implemented as, for example, a liquid crystal display, a thin film transistor-liquid crystal display, an organic light-emitting diode, a flexible display, a 3D display, an electrophoretic display, or the like. Also, according to an implementation form of the medical imaging device 100a, the medical imaging device 100a may include two or more displays 330.

The display 230 according to an embodiment may display a user interface for setting the plurality of regions in the raw data or the image generated from the raw data.

The display 230 according to an embodiment may display the user interface for selecting at least one reconstruction processing method for each of the plurality of set regions. For example, the display 230 may display the various reconstruction processing methods provided by the medical imaging device 100a, and may allow the user to select the at least one reconstruction processing method for each of the plurality of regions. Also, the display 230 may display a user interface for selecting at least one of various reconstruction processing algorithms corresponding to each of the reconstruction processing methods.

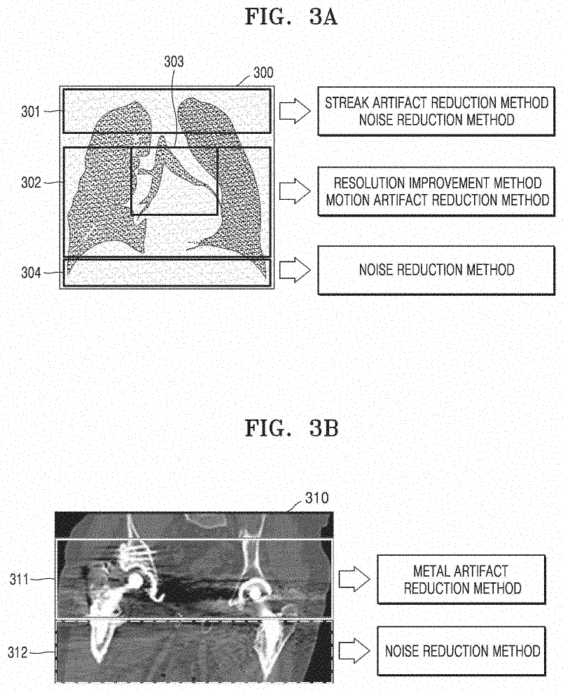

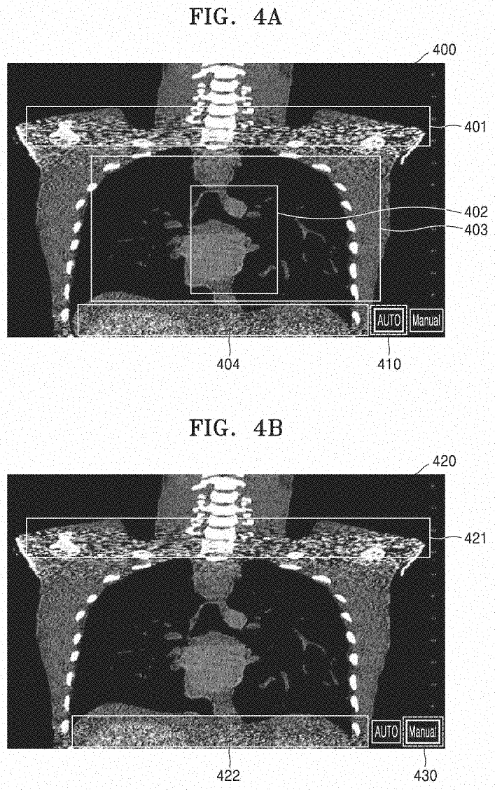

FIGS. 3A and 3B are diagrams for explaining a method of setting a plurality of regions according to an embodiment.

The medical imaging device 100a according to an embodiment may set the plurality of regions based on raw data or an image generated from the raw data. Referring to FIG. 3A, the medical imaging device 100a may obtain the data by performing CT imaging on the chest of a human body, and may generate an image 300 based on the obtained raw data. At this time, the generated image 300 may be an image generated using a reconstruction algorithm such as filtered back-projection (FBP), The image 300 obtained by imaging the chest may include a plurality of regions indicating a shoulder 301 of the human body, a lung 302, a heart 303, an abdomen 304, etc. A type of artifact and a noise level appearing in the image 300 may be different depending on each region, For example, because components of each organ of the human body are different from each other, the characteristics that appear when X-rays are transmitted may be different, and thus the type of artifact and the noise level appearing in the image 300 may be different. Accordingly, in order to more effectively improve the image quality of the image 300, a method of applying different reconstruction processing methods according to each region is required.

For example, referring to FIG. 3A, a streak artifact may appear in the first region 301 representing the shoulder in the image 300 due to a structure such as the shoulder, a bone, and the like, and the noise level may be high. The region 302 representing the lung in the image 300 may have a lower resolution relative to regions representing other organs and a motion artifact may appear by respiration. In a region representing the heart in the image 300, a motion artifact may appear by a heartbeat. Also, a metal artifact may appear in a region including a metal in the image 300. Therefore, in order to improve the image quality of the image 300, the medical imaging device 100a may apply at least one of a streak artifact reduction method, a motion artifact reduction method, a metal artifact reduction method, a noise reduction method, or a resolution improvement method.

When at least one reconstruction processing method is uniformly applied to all regions of the image 300, unnecessary image processing may be applied according to a region, and thus an amount of computation may be excessively increased. Therefore, the medical imaging device 100a according to an embodiment may set a plurality of regions based on the raw data or the image generated from the raw data, and individually perform at least one reconstruction method necessary for each region based on the image characteristics of each region.

For example, referring to FIG. 3A, the medical imaging device 100a may apply the streak artifact reduction method and the noise reduction method to the first region 301 representing the shoulder, the resolution improvement method and the motion artifact reduction method to the second region 302 representing the lung, and the noise reduction method to the third region 304 representing the abdomen, respectively. Accordingly, the medical imaging device 100a may prevent an unnecessary algorithm from being applied to each of the plurality of regions, and may reduce an amount of computation compared to a case where the reconstruction processing method is applied to all the regions in the same manner.

As another example, referring to FIG. 3B, the medical imaging device 100a may generate an image 310 from raw data obtained by imaging a patients pelvis. At this time, the generated image 310 may mean an image before various image processing for improving the image quality of a CT image is applied. For example, the image 310 may be a reconstructed image using a reconstruction algorithm such as filtered back projection (FBP).

Referring to FIG. 3B, the image 310 may represent a metal included in an object, and a metal artifact may appear in a region 311 including the metal. At this time, the medical imaging device 100a may apply the metal artifact reduction method only to the region 311 including the metal. For example, as shown in FIG. 36, the medical imaging device 100a may apply the metal artifact to the region 311 including the metal and the noise reduction method to a region 312 that does not include a metal. Accordingly, the medical imaging device 100a may more efficiently generate a CT image with improved image quality. Also, the medical imaging device 100a according to an embodiment may generate the CT image with improved image quality more quickly by applying at least one reconstruction processing method in parallel to each of the plurality of regions.

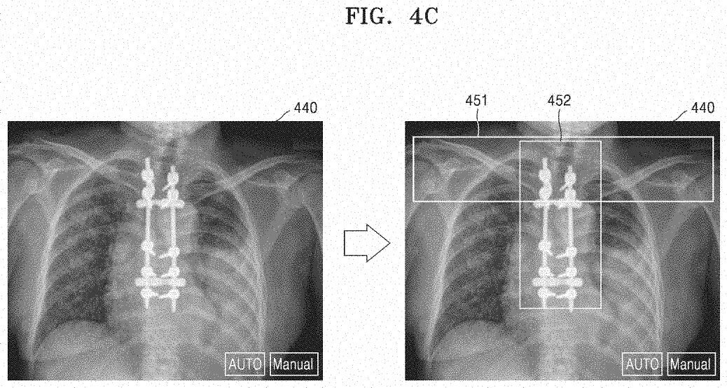

FIGS. 4A to 4C are diagrams for explaining a process performed by the medical imaging device of setting a plurality of regions according to an embodiment.

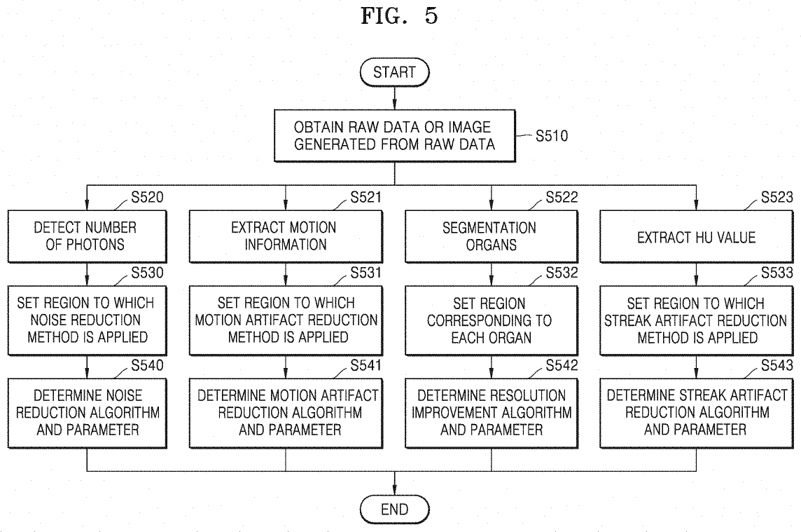

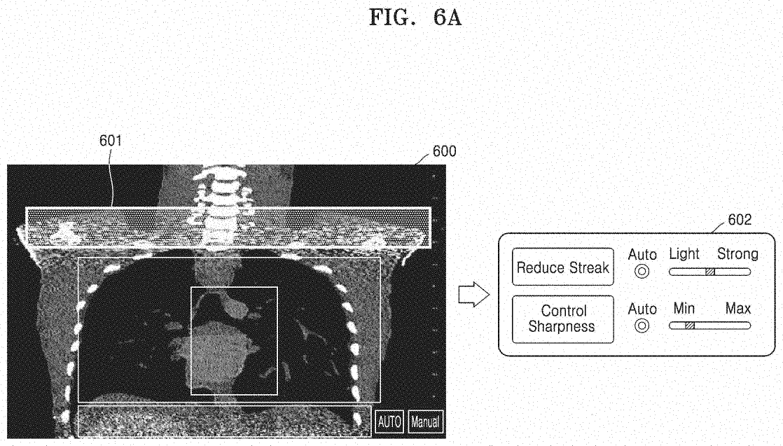

The medical imaging device 100a according to an embodiment may set the plurality of regions based on raw data or an image generated from the raw data. For example, the medical imaging device 100a may automatically set the plurality of regions based on the anatomical characteristics of an object. Referring to FIG. 4A, the medical imaging device 100a may set a first region 401 representing a shoulder, a second region 402 representing a heart, a third region 403 representing a lung, and a fourth region 404 representing an abdomen in an image 400 generated from the raw. A reference by which the medical imaging device 100a automatically sets the plurality of regions may be different according to an embodiment. Various references for automatically setting the plurality of regions will be described later with reference to FIG. 5.

According to another embodiment, the medical imaging device 100a may manually set the plurality of regions. For example, referring to FIG. 4B, a user may wish to read a first region 421 representing the shoulder more accurately in an image 420 generated from the raw data. When a streak artifact appears in the first region 421, the medical imaging device 100a needs to improve the image quality by applying a streak artifact reduction method to the first region 421. At this time, the medical imaging device 100a may receive an external input for setting the first region 421 in the image 420. The medical imaging device 100a may improve the image quality of the first region 421 by applying the streak artifact reduction method only to the first region 421 in response to the received external input.

Also, the user may wish to reduce a noise level of the fourth region 422 representing the abdomen in the image 420. The user may set the fourth region 422 as one region and apply a noise reduction method to the fourth region 422. At this time, the medical imaging device 100a may perform the streak artifact reduction method applied to the first region 421 and the noise reduction method applied to the fourth region 422 in parallel.

According to another embodiment, the medical imaging device 100a may set the plurality of regions based on a scout image. For example, referring to FIG. 40, the medical imaging device 100a may set the plurality of regions based on a scout image 440 obtained before CT imaging the object to obtain a final CT image. Because the scout image 440 indicates the internal structure of the object, the user may easily set the plurality of regions to which different reconstruction processing is applied based on the scout image 440. For example, referring to FIG. 4C, the medical imaging device 100a may set the plurality of regions based on an external input for selecting a region 451 representing a shoulder and a region 452 including a metal in the scout image 440.

The medical imaging device 100a according to an embodiment may display a user interface for setting the plurality of regions automatically or manually. For example, referring to FIG. 4A, the medical imaging device 100a may automatically set the plurality of regions in response to an external input for selecting a menu 410 “Auto”. For example, when the display 230 is implemented as a touch screen, the external input for selecting the menu 410 “Auto” may include an input for touching the menu 410 “Auto”.

As another example, referring to FIG. 4B, the medical imaging device 100a may manually set the plurality of regions in response to an external input for selecting a menu 430 “Manual”. For example, the medical imaging device 100a may manually set the plurality of regions by receiving an input for dragging predetermined regions 421 and 422 in the image 420 generated from the raw data,

FIG. 5 is a diagram for explaining a process performed by the medical imaging device of automatically setting a plurality of regions according to an embodiment.

In operation S510, the medical imaging device 100a may obtain raw data or an image generated from the raw data.

In operation S520, the medical imaging device 100a may measure the number of photons detected by a detector. The medical imaging device 100a may determine a noise level corresponding to a specific region based on the number of detected photons. In operation S530, the medical imaging device 100a may set a region in which the number of photons detected by the detector is equal to or less than a threshold value as one region. In operation S540, the medical imaging device 100a may determine a noise reduction algorithm and an algorithm parameter to be applied to the set region.

In operation S521, the medical imaging device 100a may extract motion information based on the raw data. For example, the medical imaging device 100a may calculate a motion vector based on the raw data corresponding to angle periods facing each other, and extract the motion information using the calculated motion vector. At this time, the motion information may include, but not limited to, forms such as a motion map, a motion index, a motion vector field (MVF), and the like.

In operation S531, the medical imaging device 100a may set, as one region, a region in which an occurrence degree of a motion artifact is equal to or higher than a threshold level, based on the extracted motion information. In operation S541, the medical imaging device 100a may determine a motion artifact reduction algorithm and an algorithm parameter to be applied to the set region.

In operation S522, the medical imaging device 100a may segment organs of a human body appearing in the image generated from the raw data. In operation S532, the medical imaging device 100a may set a region corresponding to each organ as one region based on the segmented organs. For example, the medical imaging device 100a may set an region representing a shoulder, an region representing a heart, an region representing a lung, and an region representing an abdomen in different regions in the image generated from the raw data. In operation S542, the medical imaging device 100a may determine a resolution improvement algorithm and an algorithm parameter to be applied to the set region.

In operation S523, the medical imaging device 100a may extract a Hounsfield unit (HU) value of pixels constituting the image generated from the raw data. In operation S533, the medical imaging device 100a may automatically detect a region where an occurrence degree of a streak artifact is equal to or higher than the threshold level based on the extracted HU value, and set the detected region as one region. In operation S543, the medical imaging device 100a may determine a streak artifact reduction algorithm and an algorithm parameter to be applied to the set region.

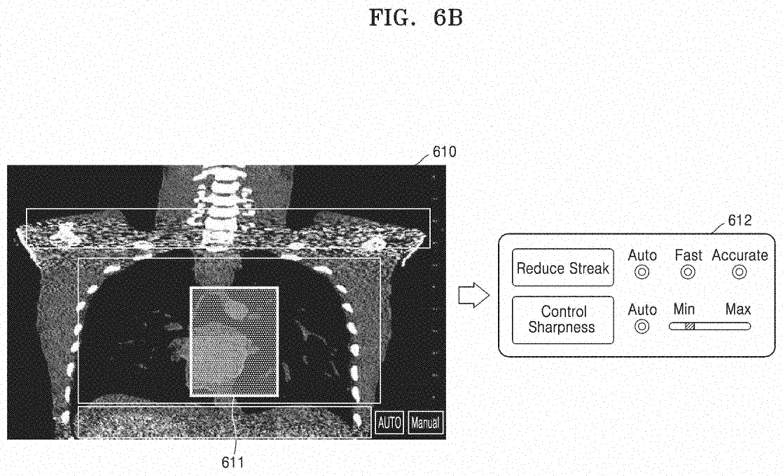

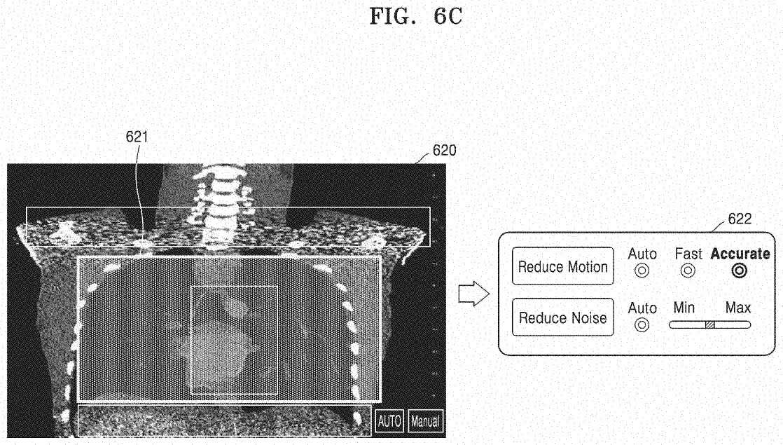

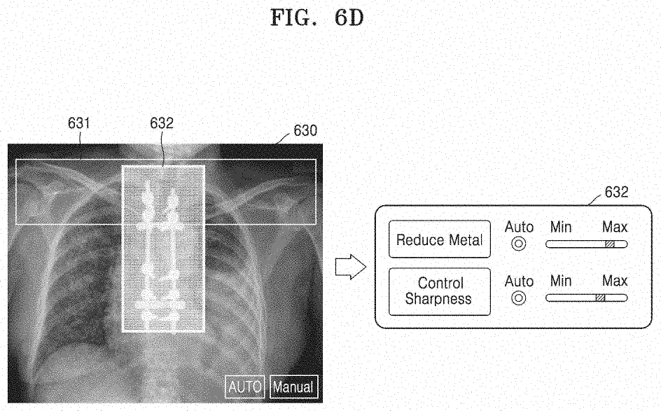

FIGS. 6A to 6D are diagrams for explaining a process of setting a parameter of a reconstruction processing method differently according to an embodiment.

The medical imaging device 100a according to an embodiment may automatically set a plurality of regions according to a predetermined reference of the medical imaging device 100a and automatically determine at least one reconstruction processing method applied to each of the plurality of regions. However, even in the above-described case, the medical imaging device 100a may allow a user to change at least one of a type or a parameter of the reconstruction processing method applied to each of the plurality of regions as required. For example, the parameter of the reconstruction processing method may indicate an application level of the reconstruction processing method. Accordingly, the medical imaging device 100a may reconstruct a CT image having image quality desired by the user.

For example, referring to FIG. 6A, the user may wish to change a parameter of a reconstruction processing method applied to a first region 601 representing a shoulder in an image 600 generated from the raw data. For example, when it is determined that a level of a streak artifact appearing in the first region 601 is equal to or higher than the threshold level, the user may wish to increase the application level of a streak artifact reduction method applied to the first region 601.

The medical imaging device 100a according to an embodiment may display a user interface 602 for changing the parameter of the reconstruction processing method. For example, the medical imaging device 100a may display a user interface representing at least one of the type or the parameter of the automatically determined reconstruction processing method. For example, referring to FIG. 6A, the medical imaging device 100a may display the application level of the reconstruction processing method applied to the first region 601 as the graphic user interface (GUI) 602 in the form of a scroll bar. For example, the medical imaging device 100a may automatically determine a streak artifact reduction method and a sharpness improvement method as the reconstruction processing method applied to the first region 601. At this time, the medical imaging device 100a may display the user interface 602 representing parameters of the streak artifact reduction method and the sharpness improvement method. As shown in FIG. 6A, the medical imaging device 100a may express the application level of the reconstruction processing method as “Light” and “Strong”, or “Min” and “Max”, but is not limited thereto.

The medical imaging device 100a according to an embodiment may change the parameter of the reconstruction processing method applied to the first region 601 in response to an external input received through the user interface 602. For example, the medical imaging device 100a may change the parameter of the reconstruction processing method applied to the first region 601 in response to an external input for moving the scroll bar to the left and right.

As another example, the user may wish to change a parameter of the reconstruction processing method applied to a second region representing the heart in the image generated from the raw data. Referring to FIG. 6B, in response to an external input for selecting a second region 611 in an image 610, the medical imaging device 100a may display a user interface 612 indicating at least one of a type or a parameter of the reconstruction processing method applied to the second region 611. For example, the medical imaging device 100a may display a motion artifact reduction method and a resolution improvement method applied to the second region 611 according to an internal instruction, and may display the user interface 612 indicating parameters of the motion artifact reduction method and the resolution improvement method.

The medical imaging device 100a according to an embodiment may change the parameters of the motion artifact reduction method and the resolution improvement method in response to an external input received through the user interface. For example, referring to FIG. 6B, in order to more accurately correct a motion of an object, the user may select a mode “Accurate”. Alternatively, when it is desired to generate a CT image with improved image quality faster, the user may select a mode “Fast”. When the mode “Accurate” is selected, the motion of the object may be corrected more accurately, but the speed may be slowed down because an amount of computation is greater than that in the mode “Fast”. Alternatively, when the mode “Fast” is selected, the CT image with improved image quality may be generated faster, but the effect of reducing motion artifacts may be relatively low. Therefore, the user may change the parameter of the motion artifact reduction method as needed.

The user may wish to change a parameter of the reconstruction processing method applied to a third region indicating the lung in the image generated from the raw data. Referring to FIG. 6C, in response to an external input for selecting a third region 621 in an image 620, the medical imaging device 100a may display a user interface 622 representing at least one of a type or a parameter of the reconstruction processing method applied to the third region 621. For example, the medical imaging device 100a may display a motion artifact reduction method and a noise reduction method applied to the second region 611 according to an internal instruction, and may display the user interface 622 indicating parameters of the motion artifact reduction method and the noise reduction method. Then, the medical imaging device 100a may change the parameters of the motion artifact reduction method and the noise reduction method in response to an external input received through the user interface.

As another example, the user may wish to change a parameter of the reconstruction processing method applied to a region including a metal in a scout image. For example, referring to FIG. 6D, in response to an external input for selecting a region 631 including the metal in a scout image 630, the medical imaging device 100a may display a user interface 632 representing at least one of a type or a parameter of the reconstruction processing method applied to the region 631 including the metal. The medical imaging device 100a may then change the parameter of the reconstruction processing method applied to the region 631 including the metal in response to an external input received through the user interface 632.

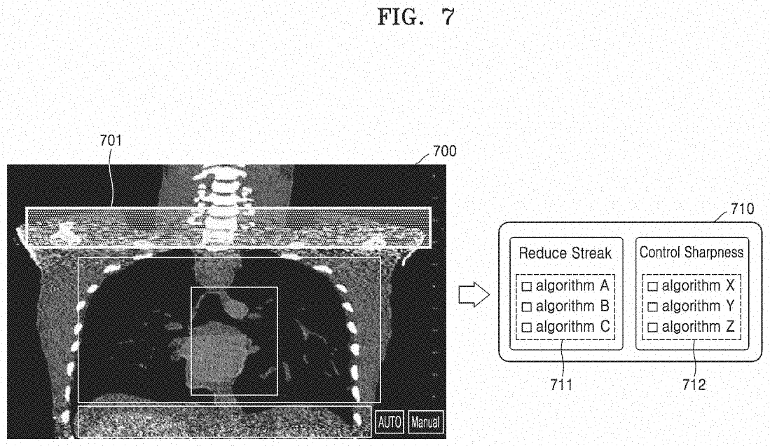

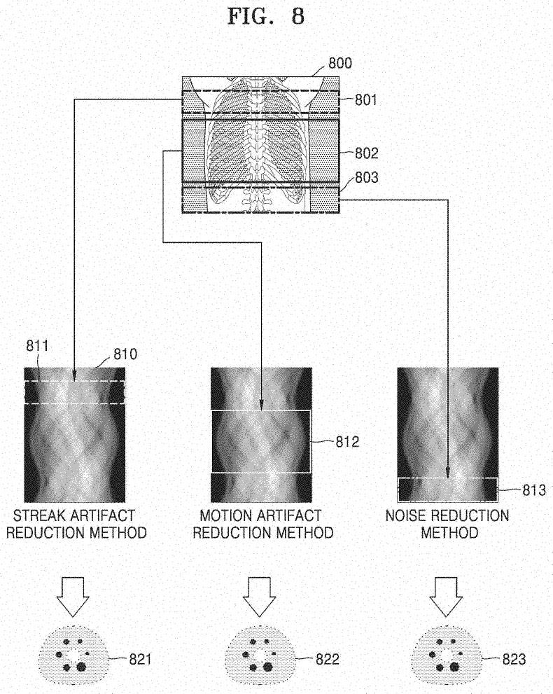

FIG. 7 is a diagram for explaining a process of manually selecting a reconstruction processing algorithm applied to each of a plurality of regions according to an embodiment.

The medical imaging device 100a according to an embodiment may receive an input that selects at least one reconstruction processing algorithm for each of the plurality of regions and generate a CT image using at least one reconstruction processing algorithm selected in response to the received input.

For example, referring to FIG. 7, a streak artifact reduction method and a sharpness improvement method may be determined as a reconstruction processing method applied to a region 701 representing a shoulder in an image 700 generated from raw data. At this time, the medical imaging device 100a may allow a user to select a preferred algorithm from among various reconstruction processing algorithms corresponding to the streak artifact reduction method and the sharpness improving method, respectively.

According to an embodiment, the medical imaging device 100a may display a user interface 710 for selecting one of the various reconstruction processing algorithms. For example, the medical imaging device 100a may display an algorithm list 711 corresponding to the streak artifact reduction method and an algorithm list 712 corresponding to the sharpness improving method, and allow the user to select a desired algorithm from the displayed algorithm lists 711 and 712.

FIG. 8 is a diagram for explaining a process of applying at least one reconstruction processing method to each of a plurality of regions according to an embodiment.

The medical imaging device 100a according to the embodiment may apply at least one reconstruction processing method to each of the set plurality of regions. For example, the medical imaging device 100a may apply at least one determined reconstruction processing method to raw data corresponding to each of the plurality of regions.

Referring to FIG. 8, the medical imaging device 100a may set a first region 801 representing a shoulder, a second region 802 representing a lung, and a third region 803 representing an abdomen in an image 800 generated from the raw data. The medical imaging device 100a may apply a streak artifact reduction method to the first region 801, a motion artifact reduction method to the second region 802, and a noise reduction method to the third region 803. The medical imaging device 100a may extract first raw data 811 corresponding to the first region 801 from the entire raw data obtained by imaging an object and apply the streak artifact reduction method to the first raw data 811 to reconstruct a CT image 821 corresponding to the first region 801, The medical imaging device 100a may extract second raw data 812 corresponding to the second region 802 from the entire raw data and apply the motion artifact reduction method to the second raw data 812 to reconstruct a CT image 822 corresponding to the second region 802. The medical imaging device 100a may extract third raw data 813 corresponding to a third region 803 from the entire raw data and apply the noise reduction method to the third raw data 813 to reconstruct a CT image 823 corresponding to the third region 803.

According to another embodiment, the medical imaging device 100a may set a region representing a lung and a region representing a heart in the image 800 generated from the raw data. Then, the medical imaging device 100a may determine the motion artifact reduction method as a reconstruction processing method applied to the region representing the lung, and determine the noise reduction method as a reconstruction processing method applied to the region representing the heart, At this time, raw data corresponding to the region representing the lung and raw data corresponding to the region representing the heart may overlap with each other. The medical imaging device 100a may apply the motion artifact reduction method to the raw data corresponding to the region representing the lung and apply the noise reduction method to the raw data corresponding to the region representing the heart, and then may output a CT image in which the noise reduction method is applied to an overlapped region. Alternatively, according to an embodiment, the medical imaging device 100a may output two types of CT images.

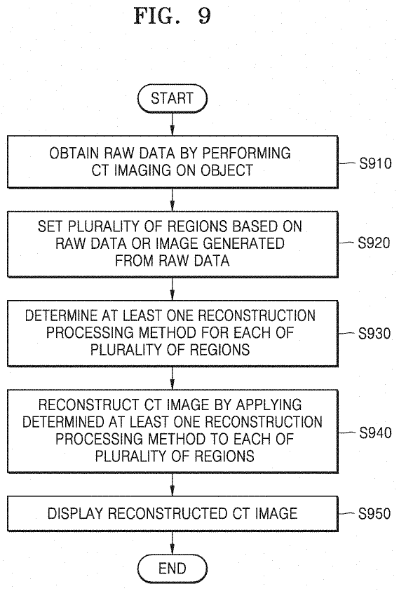

FIG. 9 is a flowchart illustrating a medical image processing method according to an embodiment.

In operation S910, the medical imaging device 100a may obtain raw data generated by performing CT imaging on an object. The raw data may be obtained in various manners such as being obtained from a scanner of the medical imaging device 100a, received from an external device, or the like.

In operation S920, the medical imaging device 100a sets a plurality of regions based on the raw data or an image generated from the raw data.

The plurality of regions may be regions requiring different reconstruction processing methods. According to an embodiment, the plurality of regions may be regions that are distinguished according to the anatomical characteristics of the object. For example, the medical imaging device 100a may set the plurality of regions by segmenting organs of a human body.

According to an embodiment, the medical imaging device 100a may automatically set the plurality of regions based on the anatomical characteristics of the object.

In operation S930, the medical imaging device 100a determines at least one reconstruction processing method for each of the plurality of regions.

The reconstruction processing method may include a streak artifact reduction method, a motion artifact reduction method, a metal artifact reduction method, a resolution improvement method, a noise reduction method, etc. and each reconstruction processing method may be implemented by various algorithms.

The medical imaging device 100a according to an embodiment may determine a parameter of at least one reconstruction processing method applied to each of the plurality of regions based on the image characteristic of each of the plurality of regions. For example, the medical imaging device 100a may determine the parameter of the at least one reconstruction processing method applied to each of the plurality of regions based on at least one of a degree of a noise level, an occurrence degree of a motion artifact, an occurrence degree of a streak artifact, or an occurrence degree of a metal artifact.

The medical imaging device 100a according to an embodiment may determine at least one reconstruction processing algorithm for applying the at least one reconstruction processing method to each of the plurality of regions, For example, the medical imaging device 100a may provide a plurality of algorithms for applying the streak artifact reduction method, the metal artifact reduction method, the motion artifact reduction method, the noise reduction method, and the resolution improvement method, respectively. The medical imaging device 100a may automatically determine one of the plurality of algorithms according to the initial setting of the medical imaging device 100a, Alternatively, according to an embodiment, the medical imaging device 100a may determine one of the plurality of algorithms based on a preference of a user, but is not limited thereto.

According to another embodiment, the medical imaging device 100a may receive an input that selects at least one reconstruction processing algorithm for each of the plurality of regions. The medical imaging device 100a may apply the at least one selected reconstruction processing algorithm to each of the plurality of regions in response to the received input.

The medical imaging device 100a according to an embodiment may change the at least one automatically determined reconstruction processing method in response to an external input. As described above, the medical imaging device 100a may automatically determine the at least one reconstruction processing method for each of the plurality of regions. However, the medical imaging device 100a may allow the user to change at least one of a type or a parameter of the reconstruction processing method applied to each of the plurality of regions as needed.

In operation S940, the medical imaging device 100a reconstructs a CT image by applying the determined reconstruction processing method to each of the plurality of regions.

According to an embodiment, the medical imaging device 100a may reconstruct the CT image by performing reconstruction processing on each of the plurality of regions in parallel, thereby reconstructing the CT image more quickly.

In operation S950, the medical imaging device 100a displays the reconstructed CT image.

The above-described embodiments of the present disclosure may be embodied in form of a computer-readable recording medium for storing computer executable instructions and data. The instructions may be stored in form of program codes and, when executed by a processor, may perform a certain operation by generating a certain program module. Also, when executed by a processor, the instructions may perform certain operations of the disclosed embodiments.

While embodiments of the present disclosure have been particularly shown and described with reference to the accompanying drawings, it will be understood by those of ordinary skill in the art that various changes in form and details may be made therein without departing from the spirit and scope of the invention as defined by the appended claims. The disclosed embodiments should be considered in descriptive sense only and not for purposes of limitation.

1. A medical imaging device comprising:
a data obtainer configured to obtain raw data by performing computed tomography (CT) imaging on an object;
a processor configured to automatically set a plurality of regions based on an image generated from the raw data or the raw data, anatomical characteristics of the object, and at least one artifact appearing in the image, determine at least one reconstruction processing method differently for each of the plurality of regions based on a type of artifact and anatomical characteristics appearing in each of the plurality of regions, and reconstruct a CT image by applying the determined at least one reconstruction processing method to each of the plurality of regions; and
a display displaying the reconstructed CT image,
wherein the at least one reconstruction method comprises at least one of a streak artifact reduction method, a motion artifact reduction method, a metal artifact reduction method, a noise reduction method, and a resolution improvement method, or a combination thereof.

2. The medical imaging device of claim 1, wherein the processor is further configured to determine, for each of the plurality of regions, at least one reconstruction processing algorithm corresponding to the at least one reconstruction processing method, and, for each of the plurality of regions, apply the determined at least one reconstruction processing algorithm to reconstruct the CT image from the raw data.

3. The medical imaging device of claim 1, wherein the processor is further configured to automatically determine the at least one reconstruction processing method according to a predetermined criterion.

4. The medical imaging device of claim 3, wherein the processor is further configured to change the at least one automatically determined reconstruction processing method in response to an external input.

5. The medical imaging device of claim 1, wherein the processor is further configured to receive an input for selecting at least one reconstruction processing algorithm for each of the plurality of regions, and, in response to the received input, reconstruct the CT image using the selected at least one reconstruction processing algorithm.

6. The medical imaging device of claim 5,
wherein the display further displays a user interface representing at least one of a type or a parameter of a reconstruction processing algorithm provided by the medical imaging device, and
wherein the processor is further configured to receive, through the user interface, an input for selecting the at least one reconstruction processing algorithm for each of the plurality of regions.

7. The medical imaging device of claim 1, wherein the processor is further configured to perform reconstruction processing on each of the plurality of regions in parallel to reconstruct the CT image.

8. A medical image processing method comprising:
obtaining raw data by performing computed tomography (CT) imaging on an object;
automatically setting a plurality of regions based on an image generated from the raw data or the raw data, anatomical characteristics of the object, and at least one artifact appearing in the image;
determining at least one reconstruction processing method differently for each of the plurality of regions based on a type of artifact and anatomical characteristics appearing in each of the plurality of regions;
reconstructing a CT image by applying the determined at least one reconstruction processing method to each of the plurality of regions; and
displaying the reconstructed CT image,
wherein the at least one reconstruction method comprises at least one of a streak artifact reduction method, a motion artifact reduction method, a metal artifact reduction method, a noise reduction method, and a resolution improvement method, or a combination thereof.

9. The medical image processing method of claim 8, further comprising determining, for each of the plurality of regions, at least one reconstruction processing algorithm corresponding to the at least one reconstruction processing method,
wherein the reconstructing of the CT image comprises, for each of the plurality of regions, applying the determined at least one reconstruction processing algorithm to reconstruct the CT image from the raw data.

10. The medical image processing method of claim 8, wherein the determining of the at least one reconstruction method comprises, automatically determining the at least one reconstruction processing method according to a predetermined criterion.

11. The medical image processing method of claim 10, further comprising changing the at least one automatically determined reconstruction processing method in response to an external input.

12. The medical image processing method of claim 8, reconstructing of the CT image comprises,
receiving an input for selecting at least one reconstruction processing algorithm for each of the plurality of regions; and
in response to the received input, reconstructing the CT image using the selected at least one reconstruction processing algorithm.

13. The medical image processing method of claim 12, wherein receiving of the input for selecting at least one reconstruction processing algorithm for each of the plurality of regions comprises,
displaying a user interface representing at least one of a type or a parameter of a reconstruction processing algorithm provided by the medical imaging device; and
receiving, through the user interface, an input for selecting the at least one reconstruction processing algorithm for each of the plurality of regions.

14. A computer-readable recording medium having recorded thereon a computer program code for executing a medical image processing method when read and executed by a processor, the medical image processing method comprising:
obtaining raw data by performing computed tomography (CT) imaging on an object;
automatically setting a plurality of regions based on an image generated from the raw data or the raw data, anatomical characteristics of the object, and at least one artifact appearing in the image;
determining at least one reconstruction processing method differently for each of the plurality of regions based on a type of artifact and anatomical characteristics appearing in each of the plurality of regions;
reconstructing a CT image by applying the determined at least one reconstruction processing method to each of the plurality of regions; and
displaying the reconstructed CT image,
wherein the at least one reconstruction method comprises at least one of a streak artifact reduction method, a motion artifact reduction method, a metal artifact reduction method, a noise reduction method, and a resolution improvement method, or a combination thereof.

15. (canceled)
